(19) United States
(12) Patent Application Publication
(10) Pub. No.: US 2014/0303664 A1
(43) Pub. Date: Oct. 9, 2014

(54) ATRAUMATIC STERNAL PLATE

(71) Applicant: AESCULAP AG, Tuttlingen (DE)

(72) Inventors: Thomas Beck, Durchhausen (DE); Bernd Blender, Tuttlingen (DE); Andreas Elisch, Schramberg (DE); Theodor Lutze, Balgheim (DE); Pedro Morales, Tuttlingen (DE); Robert Vogtherr, Tuttlingen (DE); Jürgen Wegmann, Stockach (DE); Dieter Weisshaupt, Immendingen (DE)

(73) Assignee: AESCULAP AG, Tuttlingen (DE)

(21) Appl. No.: 14/355,717

(22) PCT Filed: Oct. 12, 2012
(86) PCT No.: PCT/EP2012/070275

§ 371 Date: May 1, 2014

(30) Foreign Application Priority Data
Nov. 2, 2011 (DE) 10 2011 117 413.7

(51) Int. Cl. A61B 17/12

(52) U.S. Cl. CPC A61B 17/12009; A61B 2017/12004
USPC 606/191

(57) ABSTRACT

The present application relates to an atraumatic sternal plate for haemostasis for a retractor with a pressure plate and a retaining device attached to the pressure plate. The pressure plate can be attached, by means of the retaining device, to either the blades of a retractor or to a sternal half of the patient, so that the pressure plate abuts the sagittal incision surface of a sternal half when spreading the sternum of a patient by means of a retractor.

The present invention relates an atraumatic sternal plate for hemostasis, and in particular to an atraumatic sternal plate that is used in conjunction with a retractor and is either applied to the retractor before the same is introduced into the opened sternum, or that is applied to one or both sternum halves before a retractor is introduced into the opened sternum.

Numerous methods are known in the prior art for spreading a patient's sternum so as to gain access to a patient's thoracic cavity and/or to the heart. A retractor typically in use today has two parallel arms that can be moved away from each other by way of a rack-and-pinion mechanism or a similar device. Two blades are customarily provided on each of the two parallel retractor arms, the incipient spacing of the retractor arms causing the blades to engage and press the sagittal incision surface of the respective sternum half, thereby spreading the sternum as the distance between the retractor arms increases. Conventional blades are not very wide, whereby the entire force is exerted on the two sternum halves over only a very small area. At the same time, the force required to spread the sternum can be considerable. As a result, bones located directly on or beneath the blades can become injured, for example chipped, and the surrounding tissue can become severely traumatized. The patient's healing period after the procedure is thus protracted, and the patient additionally suffers greater pain during this time.

One example of a retractor is shown in U.S. patent application US 2009 0,259,107 A1. This retractor comprises a single blade on each side of the bisected sternum and pressure sensors which measure the force acting on the two retractor arms so as to limit the trauma to the tissue. If the pressure sensors detect disturbances or peaks in the progression of the pressure levels, the retractor decreases the distance between the two retractor arms so as to reduce the pressure on the bones in the two sternum halves. However, if a drop in pressure occurs suddenly, it is likely due to a piece of bone having already chipped or splintered. As a result of the design, this retractor is thus not able to prevent bone material of the bisected sternum from chipping or splintering. Moreover, the design of this retractor is disadvantageous because it severely limits the field of view inside the patient available to the surgeon because the retractor is located over the sternal aperture.

Retractors acting atraumatically are also known from the prior art. The published prior art documents U.S. Pat. No. 3,882,855 A, DE 1 970 499 C2 and DE 29 702 249 U1 show flexible brain tissue retractors, which are made of pliable and dimensionally stable material enclosed by soft and elastically deformable material, wherein the deformability increases as the distance from the core material increases. Such a composition results in no, or in only minor, pressure points on the contact surface.

It was proposed in the prior art (JP 2002 102 234 A) to place a clip on the sternum halves so as to stop the bleeding of the sagittal incision surfaces of a sternal opening. This clip is engaged on the inside and outside of the sternum half in the tissue with the aid of teeth. The center part of the clip, which covers the sagittal incision surface of the sternum, can be provided with a urethane foam material intended to support hemostasis. The teeth pressed into the tissue, however, are anything but atraumatic and can cause considerable injury, in particular on the inside of the sternum half, for example by pinching or tearing or slitting the internal mammary artery (IMA). However, frequently a retractor is to be used to expose the internal mammary artery in particular, so as to dissect the same free and use it as a bypass. It is thus doubly disadvantageous if the IMA is pinched. This requires initially the spreading of the sternum to be reduced, the inserted retractor to be removed, the clips to be repositioned in the hope that the IMA is not pinched again, the retractor to be re-inserted, and the sternum to be spread again. This takes a lot of time, resulting in considerable added costs, but also adversely impacts the patient because the extended stress results in unnecessary tissue trauma. Moreover, the IMA can become damaged when pinched, whereby it may no longer be usable for a bypass or, at a minimum, the usable length of the IMA is reduced.

When a retractor that is presently state of the art is used, typically cloths or compresses are placed or pushed between the blades and the sagittal incision surface of the sternum to absorb the exiting blood and thereby stop the bleeding. However, these cloths can easily shift with a minor movement or a minor relocation of the retractor. Overall, the inserted cloths adversely affect the surgeon's field of view. In addition, the hemostatic effect of the cloths is not satisfactory.

It is thus the object of the present invention to provide a means by means of which the bleeding at the sagittal incision surfaces of the sternum can be better stopped and which do not adversely affect the surgeon's field of view. Another object of the present invention is to provide such a means which continues to perform its function even when the retractor is relocated or displaced in the sternal opening.

The object of the present invention is achieved by an atraumatic sternal plate according to claim 1. Advantageous embodiments and refinements are the subject matter of the dependent claims.

To begin with, the meanings of several terms shall be provided here. The vertical direction in the present disclosure denotes the vertical direction in the medical sense, that is, independent of the actual position of the patient (standing up, lying down), the direction from the crown to the soles, thus, with respect to the thorax and the sternum, in the direction from cranial to caudal. Relative to the atraumatic sternal plate, the proximal side denotes the side facing the sagittal incision surface of the sternum half, while the distal side is the side facing away from the sagittal incision surface and facing the other sternum half. The medial end of a leaf spring is the end located toward the center of the pressure plate.

A first aspect of the present invention relates to an atraumatic sternal plate for hemostasis for a retractor, having a pressure plate and a retaining device provided on the pressure plate. The pressure plate can be applied to the blades of a retractor by way of the retaining device so that the pressure plate is seated against the sagittal incision surface of one sternum half when a patient's sternum is spread by the retractor.

Such an atraumatic sternal plate is used as follows. Initially, the surgeon opens the patient's sternum in the sagittal plane, whereby the bisected sternum opens up a little and a certain gap forms between the halves. The atraumatic sternal plate is applied to the blades of the retractor by the retaining device, and the retractor is inserted into the opening of the sternum with the retractor arms close beside each other. Usually two atraumatic sternal plates are used so as to cover both incision surfaces of the sternum. The retractor arms are now moved apart from each other until the sternal plate is seated against the associated incision surface of the sternum. It is only now that the spreading process begins, in which the retractor arms are moved further away from each other, whereby the sternum is spread, giving the surgeon access to the inside of the chest and an unobstructed view thereof.

In this way, the atraumatic sternal plate fulfills multiple functions. It distributes the sometimes considerable pressure exerted by the retractor blades over a significantly larger contact area, and ideally over the entire surface of the sternal plate. The sternal plate is spring-elastic for this purpose and has a suitable modulus of elasticity as well as suitable dimensions. These figures cannot be stated as fixed values since they are highly dependent on the individual patient. The key factor here is that the length and the width (or the height) are adapted to the respective patient. The length should correspond as closely as possible to the length of the sternum incision, and the width (height) should correspond to at least the thickness of the chest wall, which is to say the overall thickness of the sternum and soft tissue located thereabove. However, the width also should preferably not be much greater than the thickness of the chest wall. In the case of single-piece sternal plates, the width may also vary over the length since the thickness of the chest wall may also be variable over the length of the sternum incision. The thickness of the pressure plate and the modulus of elasticity are dependent on the spreading force that is to be exerted. This force, in turn, depends on the patient's size, age and physical condition. These sizes are matched to each other so as to achieve an application of force onto the incision surface of the sternum half that is as uniform as possible over the entire surface of the sternal plate.

In addition, the atraumatic sternal plate presses against the incision surface of the respective sternum half, thus ensuring initial hemostasis. This function cannot be carried out by a blade since it is seated against only a small portion of the incision surface. Cloths placed against the blades also cannot fulfill this function. While they are able to absorb blood, they cannot exert pressure onto the incision surface beside and between the blades.

The atraumatic sternal plate can also be applied to the blades in such a way that the retractor itself is still displaceable, to a small degree, with respect to the atraumatic sternal plate even when the retractor is spreading the sternum, without the sternal plate shifting with respect to the incision surface of the sternum half. This is achieved by not rigidly fixing the sternal plate to the blades of the retractor, but by applying the same with sufficient lateral play (lateral here denotes the surface of the sternal plate).

According to one advantageous embodiment of the first aspect of the present invention, the pressure plate has an arched elongated shape so as to conform in the vertical direction to a curvature of the sagittal incision surface of the sternum half. The incision surfaces of the sternum are not absolutely rectilinear, but are slightly arched and thus have approximately the shape of a banana or of a very narrow kidney basin. This curvature can have an interfering effect in particular during the dissection of the internal mammary artery (IMA) because, when a straight sternal plate is used, a portion of the work area can be covered by the sternal plate, which in the center region (in the longitudinal direction of the sternal plate) protrudes inwardly into the thoracic cavity beyond the sagittal incision surface of the sternum. The shape of the pressure plate is advantageously adapted to the shape of the incision surface. Since the incision surface is different from one patient to the next, and has a different length and different curvature in each case, as well as sometimes having significantly varying widths (even across the sounds of the incision surface), the shape and size of a prefabricated sternal plate can conform to the incision surface only to a certain degree. For this purpose, a larger number of sternal plates must be kept in stock so as to be able cater to all potential patients, ranging from children to men and women of normal builds to obese patients, with a suitable atraumatic sternal plate.

According to a further embodiment of the first aspect of the present invention, the retaining device is affixed on the distal side of the pressure plate. Arranging the retaining device on the distal side of the pressure plate is advantageous since the force exerted by the retractor can thus be easily applied by the blades onto the pressure plate. In this case, the blades have only to push the pressure plate, and the retaining device can remain substantially free of force. In principle, the retaining device can also be affixed on the side of the pressure plate, however it must then also be able to transmit lateral forces and bending moments, if necessary, as a result of which the retaining device must have a bulkier design and thus is likely to unnecessarily restrict the surgeon's field of view.

According to a further advantageous embodiment of the first aspect of the present invention, the retaining device is composed of two leaf springs which extend essentially parallel to the pressure plate and are attached at their medial ends to the pressure plate. In this case, each blade is gently clamped between a free end of a leaf spring and the pressure plate. However, a certain ability to reposition between the sternal plate and blades can be preserved.

According to a particularly advantageous embodiment of the first aspect of the present invention, the retaining device is composed of one leaf spring, the two free ends of which extend essentially parallel to the pressure plate and the central region of which is attached to the pressure plate. The atraumatic sternal plate is particularly easy to apply to the blades of the retractor by such a retaining device. Either the sternal plate is pushed onto the blade from the free ends of the blade, wherein the blades are arranged in each case between a leaf spring and the pressure plate, or the sternal plate is first pushed laterally onto the first blade (which is to say essentially in the longitudinal direction of the sternal plate), wherein the first blade is located between the first end of the leaf spring and the pressure plate, and the sternal plate is then pushed further until the second end of the leaf spring is able to pass the second blade, and afterwards the sternal plate is pushed in the opposite direction, so that the second blade is located between the second end of the leaf spring and the pressure plate, until the sternal plate is positioned approximately symmetrically to the blades. This configuration allows the sternal plate to still be easily moved with respect to the blades, even when the sternum is spread, without any risk of sliding off the blades or off the covered incision surface.

According to another embodiment of the first aspect of the present invention, the retaining device is composed of one leaf spring which extends essentially parallel to the pressure plate and is attached at the lateral ends to the pressure plate. In this aspect, lateral sliding of the sternal plate off the blades is entirely precluded, however, it also cannot be mounted laterally onto the blades.

A second aspect of the present invention relates to an atraumatic sternal plate for hemostasis with a pressure plate and a retaining device attached to the pressure plate. The pressure plate can be applied to a sternum half by the at least one retaining device so that the pressure plate is essentially seated against a sagittal incision surface of the sternum.

Such a sternal plate is used as follows. Initially, the surgeon opens the patient's sternum in the sagittal plane, whereby the bisected sternum opens up a little and a certain gap forms between the halves. The sternal plate is now introduced into the gap and the proximal side thereof is seated against the incision surface. Depending on the embodiment of the sternal plate, the surgeon may or must first rigidly fix the sternal plate with the aid of the retaining device. In the simplest case, the retaining device is a site to which the surgeon can apply one or more adhesive strips, threads or other means of attachment that do not form part of the invention. If adhesive strips are used, the surgeon can attach the sternal plate to the surrounding tissue on the outside only, or on the outside and the inside of the sternum. If threads are used, these are attached to the retaining device, which is advantageously located in the vicinity of the outside longitudinal edge of the sternal plate. These threads are then tensioned and rigidly fixed in the lateral direction (that is, in the direction of the patient's respective arm), either by way of a clip, a weight or an adhesive strip; however, it is also conceivable to suture the thread or the sternal plate to the patient. Customarily two atraumatic sternal plates are used here as well. Thereafter, the retractor, with the retractor arms close together, is introduced into the sternal opening, and the retractor arms are moved apart until the blades are seated against the atraumatic sternal plate. Only now does the spreading process begin by moving the retractor arms further away from each other. The atraumatic sternal plates are thus pressed against the sagittal incision surfaces of the bisected sternum, whereby they achieve the effects described for the first aspect of the invention.

If a sternal plate is rigidly fixed only on the outside of the thorax by an adhesive strip or a thread, that is, in the region of the soft tissue, the plate can easily move away from the sternum as long as no force is yet exerted by the retractor on the sternal plate. This does not, however adversely affect the effectiveness of the sternal plate when the sternum is spread.

According to one advantageous embodiment of the second aspect of the present invention, the retaining device has at least one protrusion on at least one longitudinal edge of the pressure plate projecting from the proximal side of the pressure plate. The pressure plate thus has an L-shaped or even a C-shaped cross-section at this location. In the case of an L-shape, the protrusion can be located on the inside of the thorax or on the outside of the thorax. In one instance, the protrusion is seated against the inner wall of the sternum; in the other instance, it is seated against the covering soft tissue. Such a pressure plate can also be additionally fixed by way of adhesive strips or threads until the retractor is used and moved into the engaged position.

According to one advantageous embodiment of the second aspect of the present invention, the at least one protrusion extends essentially over the entire longitudinal edge of the pressure plate. The pressure plate thus has an L shape, or even a C shape, over essentially the entire length. This improves engagement with the corresponding contact surface.

According to a particularly advantageous embodiment of the second aspect of the present invention, the retaining device has at least one protrusion on each of the two longitudinal edges, so that the sternal plate has a substantially C-shaped cross-section. At least one elastically compressible component is provided on the at least one protrusion, the component pressing the sternum from the inside and/or the soft tissue above it from the outside when the sternal plate is used on the patient, thus holding the sternal plate in position. The elastically compressible component can be a compression spring (such as a coil spring, a leaf spring, or a torsion spring), a foam, or another elastic body. Since the thickness of the patient's chest wall may be variable over the length of the sternum incision, and since the thickness of the chest wall also differs from one patient to the next and only a certain number of different atraumatic sternal plates can be kept in stock, this embodiment creates an atraumatic sternal plate that can be used for a wide variety of patients. The elastically compressible component can be located on one side only, or on both sides, of the thorax, which is to say on one protrusion only or on both opposing protrusions. In practice, the most relevant is the case in which an essentially continuous protrusion is formed on the inner longitudinal edge of the pressure plate, the protrusion being seated against the inner wall of the sternum. At least one further protrusion is arranged along the outer longitudinal edge of the pressure plate; however this protrusion has a certain separation from the soft tissue above the sternum. An elastically compressible component, such as a foam rubber pad, is provided on this further protrusion facing the soft tissue and is compressed by the further protrusion and the soft tissue, whereby the first protrusion is also pressed against the inner wall of the sternum and the atraumatic sternal plate is thus held against the sternum half.

According to a further particularly advantageous embodiment of the second aspect of the present invention, the retaining device has at least one protrusion on each of the two longitudinal edges, so that the sternal plate has a substantially C-shaped cross-section. However, the pressure plate is telescopic in the transverse direction, so that the width or the height of the sternal plate can be adjusted to the thickness of the patient's sternum and the soft tissue above. In this way, no elastic component is required between one of the protrusions and the chest wall (the inner wall or outer wall or both). In this case, the protrusions on the inside and the protrusions on the outside of the thorax are seated against the thorax. The telescopic function can be configured with additional features. For example, a tension spring element (coil tension spring, elastic band, and the like) can be provided within the telescopic pressure plate or thereon, so that the opposing protrusions are pretensioned toward the soft tissue or toward the sternum and in this way clamp or retain the chest wall between them. However, the telescopic function can also be subject to friction fit, so that the pressure plate can be telescoped relatively easily, but the protrusions can still exert a certain tension on the thorax so as to hold the atraumatic sternal plate against the respective sternum half. A locking telescopic function is also conceivable, that is, the distance between the protrusions is set manually, for example by compressing the protrusions when the atraumatic sternal plate is inserted, and a lock is then activated that prevents the protrusions from moving away from each other until the lock is released again.

According to a further particularly advantageous embodiment of the two aspects of the present invention, the pressure plate is composed of at least two segments, wherein the segments can be rotated or displaced with respect to each other so as to be adapted in the vertical direction to a curvature of the sagittal incision surface of the sternum half. Such a particularly advantageous embodiment not only allows the atraumatic sternal plate to be individually adapted to the curvature of the sternum, but also, in conjunction with the protrusions, particularly good uniform support of the same can also be ensured. In a further combination with the telescopic pressure plate, the end face of the sternum half can now be excellently covered, and the atraumatic sternal plate can essentially fully enclose the sternum half along the inner wall and the outer wall of the thorax. This not only absolutely minimizes the effect on the surgeon's field of view and excellently attaches the sternal plate to the thorax, but also improves hemostasis and thus the entry of blood into the operating area because the blood would now have to first flow around the closely seated protrusions. If the pressure plate is segmented, the protrusions are also segmented, of course, and the telescopic property then applies to each individual segment. The atraumatic sternal plate can thus also conform to the variable thickness of the thorax over the length of the sternal opening. A pressure plate having segments that can be rotated with respect to each other can be configured in a variety of ways. A type of tongue-and-groove system can be produced for this purpose, in which a portion of a segment is held rotatably on or in an adjoining segment. In this case, the tongue and grooves run perpendicularly to the surface of the pressure plate. However, it is also possible to provide individual joining members between the individual segments, the members being attached rotatably on one segment or on both segments, or the joining members can be bendable in the desired direction and flexurally rigid in the incision surface of the sternum half. It is also possible to use a continuous flat metal strip, which extends along the longitudinal direction of the pressure plate and to which the segments are attached and which is positioned perpendicularly to the surface of the pressure plate. A displacement of the individual segments can likewise be created by way of a tongue-and-groove connection, except that in this case the tongue and grove in the surface of the pressure plate run transversely to the pressure plate. Despite the rotation of the segments with respect to each other, the entire pressure plate still has the required flexural rigidity with respect to the incision surface. It is also possible to use combinations of the above-described techniques. However, it is important that the shape be capable of being adapted in the plane of the pressure plate, while the pressure plate as a whole maintains the desired flexural rigidity relative to the surface of the pressure plate.

According to a further embodiment of the two aspects of the present invention, the rotation or displacement of the segments with respect to each other is subject to such friction that the arrangement of the segments cannot be altered solely by the effect of their own gravity. This means that the surgeon can easily manually set the positions of the individual segments of the pressure plate with respect to each other, but that these are not attached to each other as loosely as chain links are, for example; instead, the system is rigid to the extent that it maintains its shape even if it no longer rests against the sagittal incision surface of the sternum.

According to a further embodiment of the two aspects of the present invention, the proximal side of the pressure plate is at least partially covered by a means for hemostasis, in particular by a collagen non-woven fabric. In this way, yet another hemostatic effect is achieved in addition to the physical hemostasis via pressure on the bleeding incision surface. However, there are numerous other options for applying a hemostatic means to the proximal side of the pressure plate or to the segments thereof. In principle, there are biological and chemical agents. Biological agents that can be applied include collagen, elastin, gelatin or sugar (cellulose), and the derivatives and hydrolysates thereof. Chemical agents include, for example, foamed PUR (polyurethane) or other plastic materials, in particular in the foamed state. However, it is also possible to use a mixture of biological and/or chemical agents. Other known agents for hemostasis are waxes, in particular bone wax (frequently a mixture of sterilized beeswax and petroleum jelly), and various textiles. All of these agents can be applied as foams, as a mesh or netting, as a woven fabric, a non-woven fabric or a knitted fabric, or can be introduced into such structures or applied thereon. These structures can be detachably or permanently applied to the pressure plate by way of an adhesive bond, adhesion, Velcro fastening or the like.

According to a further particularly advantageous embodiment of the two aspects of the present invention, the means for hemostasis is composed of a woven fabric, a non-woven fabric and/or another flat component, which is preferably covered or saturated with a chemical anticoagulant.

According to a further advantageous embodiment of the two aspects of the present invention, the pressure plate has at least one perforation and/or predetermined breaking point, by way of which at least one dimension of the pressure plate can be adapted to the size and shape of the patients' sternum or the sagittal incision surface of the sternum. For example, the predetermined breaking point can be formed of one or more embossed notches provided on one side or on both sides of the pressure plate. As an alternative, the predetermined breaking point can also be formed by one or more notches formed directly during manufacture of the pressure plate or of the segments thereof. Embossed predetermined breaking points are suitable for pressure plates made of metal, for example stainless steel, titanium or chrome steel. Predetermined breaking points that are formed directly with the pressure plate are suitable in particular for pressure plates made of plastic material, such as PEEK (polyether ether ketone) or PUR (polyurethane). In principle, perforations can also be formed directly during the manufacture of the pressure plate, in particular when pressure plates are made of plastic material; however, they can also be produced later by stamping.

According to a particularly advantageous embodiment of the two aspects of the present invention, at least one perforation and/or predetermined breaking point is provided between individual segments of the pressure plate. In this case, the perforation or predetermined breaking point is located on a protrusion of a segment, which connects this segment to the adjoining segment, or on a joining component of two segments. A few options are described above, which can be used to adapt the width or height of the atraumatic sternal plate to the thickness of the patient's chest wall. The at least one perforation and/or predetermined breaking point can also be used to adapt the length of the atraumatic sternal plate to the length of the sagittal incision. It is particularly advantageous with segmented pressure plates if the individual segments can be removed to adjust the length. Separation between the individual segments is the best option, in particular with segments that are telescopic in the direction of width or height. In the case of single-piece pressure plates, or pressure plates having only very few segments, which perhaps also cannot be adjustable in width or height, it may also be useful to provide a perforation and/or breaking point within the individual segments, or on the single segment. In particular with single-piece pressure plates, but not only with these, the at least one perforation and/or predetermined breaking point can also be used to set or adapt the width or height of the pressure plate. The perforations and/or predetermined breaking points can thus run in the longitudinal direction and in the transverse direction of the atraumatic sternal plate; however, they may also run diagonally or consist of webs having (multiple) curves. In the case of plastic pressure plates, it may also be sufficient if reference lines are printed onto one side of the pressure plate, which are helpful when cutting the pressure plate to size.

A suitable device can be prepared to adapt the shape and size of the pressure plate, depending on the material of the pressure plate. For example, forceps may be provided with a jaw shape matching certain perforations or predetermined breaking points on the pressure plate. The same applies to predetermined breaking points for severing entire segments. In this case, the device can be adapted to the connecting means or the segment protrusions or appendages. As far as handling is concerned, the length of the atraumatic sternal plate is adapted or set before the plate is applied to the blades of the retractor or to the corresponding sternum half, and optionally also before the width or height thereof is adapted by a telescopic unit in the pressure plate. If the height and/or width, and length of the atraumatic sternal plate are adapted by way of the at least one perforation and/or predetermined breaking point, the sequence of the adjustment between the length and height, or width, is generally not crucial; however, it is useful to first adapt the length, and then to adapt the height or width.

The exemplary embodiments of the present invention are described hereafter in detail with reference to the figures.

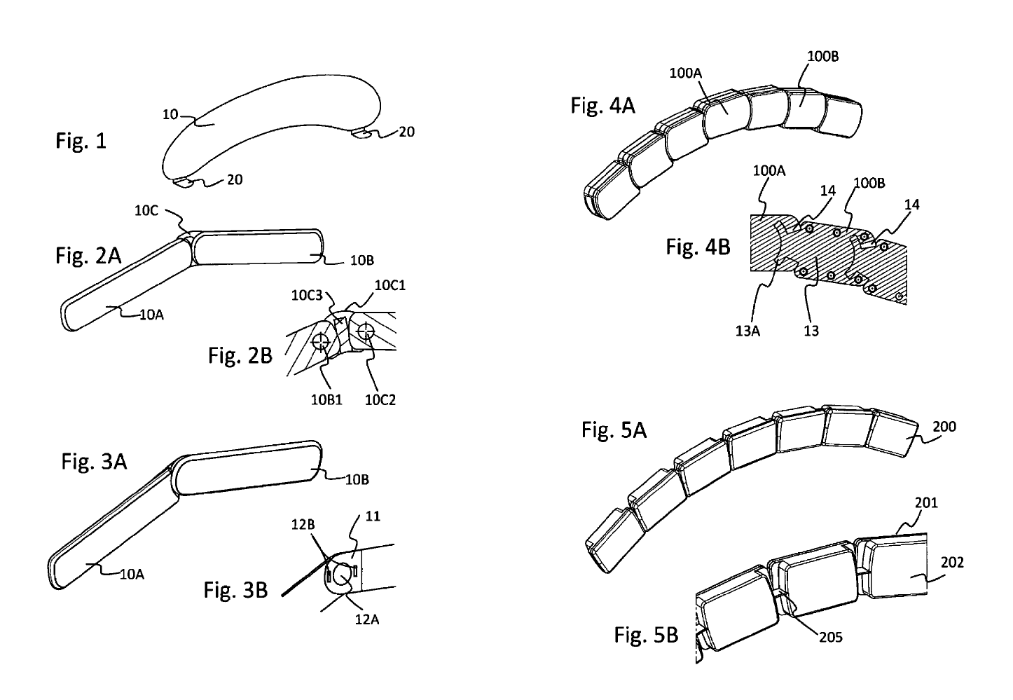

FIG. 1 shows a perspective view of a first exemplary embodiment of the present invention;

FIG. 2A shows a perspective view of a second exemplary embodiment of the present invention;

FIG. 2B shows a view of a detail from FIG. 2A;

FIG. 3A shows a perspective view of a third exemplary embodiment of the present invention;

FIG. 3B shows a view of a detail from FIG. 3A;

FIG. 4A shows a perspective view of a fourth exemplary embodiment of the present invention;

FIG. 4B shows a view of a detail from FIG. 4A;

FIG. 5A shows a perspective view of a fifth exemplary embodiment of the present invention;

FIG. 5B shows a view of a detail from FIG. 5A;

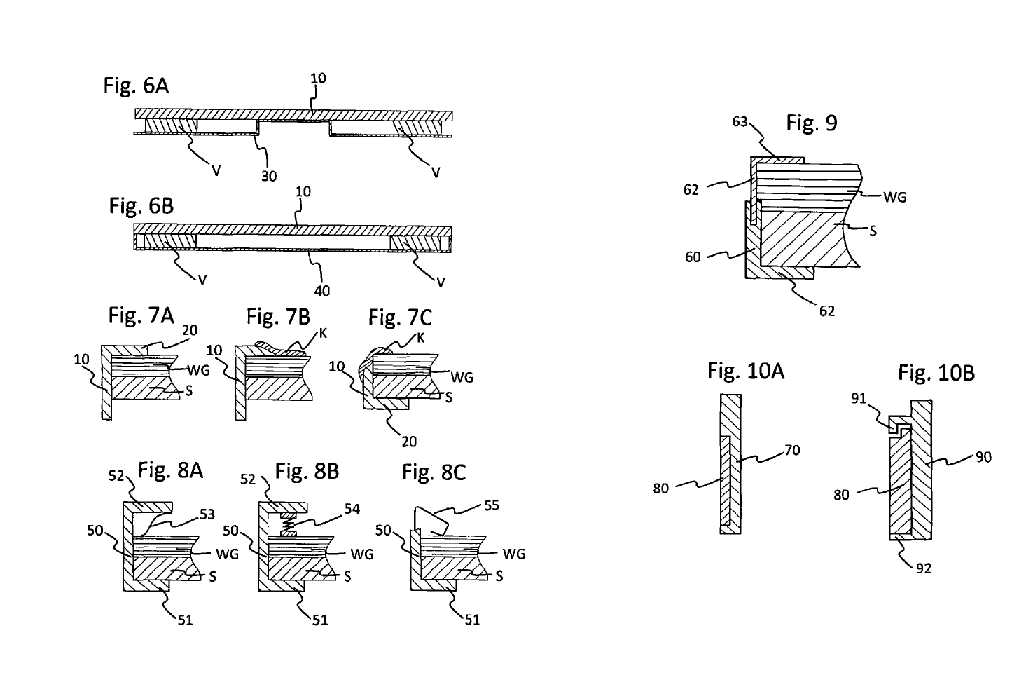

FIGS. 6A and 6B show cross-sectional views of retaining devices for applying the atraumatic sternal plate to the blades of a retractor;

FIGS. 7A, 7B and 7C show cross-sectional views of retaining devices for applying the atraumatic sternal plate to a patient's sternum;

FIGS. 8A, 8B and 8C show cross-sectional views of additional retaining devices for applying the atraumatic sternal plate to a patient's sternum;

FIG. 9 shows a cross-section of a sixth exemplary embodiment of the present invention; and

FIGS. 10A and 10B show cross-sectional views of a pressure plate having a means for hemostasis applied thereto.

A first exemplary embodiment of the present invention is described hereafter with reference to FIG. 1.

The atraumatic sternal plate for hemostasis for a retractor according to the first exemplary embodiment has a pressure plate 10 and a retaining device 20 provided on the pressure plate 10. The retaining device 20 requires two protrusions on the distal side of the pressure plate which are designed to be seated against the inner width of the sternum. The pressure plate 10 can be applied to a sternum half by way of the at least one retaining device so that the pressure plate 10 is essentially seated against a sagittal incision surface of the sternum. The atraumatic sternal plate can additionally be fixed to the patient's thorax with an adhesive strip. In this first exemplary embodiment, the atraumatic sternal plate does not include a hemostatic agent, but a woven fabric holding such an agent can be applied to the pressure plate by adhesion. On the atraumatic sternal plate according to this exemplary embodiment, the pressure plate additionally has an arched elongated shape so as to conform in the vertical direction to a curvature of the sagittal incision surface of the sternum half.

This atraumatic sternal plate allows the pressure typically exerted by the blades of a retractor on small areas of the sagittal incision surface on the patient's sternum to be distributed over the entire sagittal incision surface. As a result, stress on these areas is drastically reduced. Moreover, the pressure plate is pressed against the sagittal incision surface so that bleeding from the sagittal incision surface is reduced, if not stopped entirely. Due to the curvature of the pressure plate, in particular the adaptation of the shape of the pressure plate to the shape of the sagittal incision surface on the inside of the sternum, the surgeon has a good view of the patient's thorax, and also of the internal mammary artery in particular.

One modification of the first exemplary embodiment is that the atraumatic sternal plate is not configured with a retaining device for applying the sternal plate to the patient's sternum, but with a retaining device for applying the sternal plate to the blades of a retractor. In this case, the pressure plate 10 has a retaining device 30 as shown in FIG. 6A. A leaf spring 30 is provided on the distal side of the pressure plate 10, the spring clamping the blades V of the retractor between the spring and the pressure plate 10 if the atraumatic sternal plate is attached to a retractor. The leaf spring 30 is attached to the pressure plate 10 in the central region of the same and has two free ends which extend in the lateral direction of the pressure plate 10. However, as is shown in FIG. 6B, a leaf spring 40 can also be provided as a retaining device, which is attached at the two lateral ends thereof to the distal side of the pressure plate. If the pressure plate is composed of multiple segments, the leaf spring can be split at the center in accordance with FIG. 6A, and each of the two parts can be individually provided on one of the segments. However, it is also possible to split the leaf spring according to FIG. 6B at the center, and the two parts can be provided on individual segments of the pressure plate. In this case, it may be advantageous if the length of the leaf springs is shorter in the longitudinal direction of the pressure plate.

A second exemplary embodiment of the present invention is described hereafter with reference to FIGS. 2A and 2B.

According to the second exemplary embodiment of the present invention, an atraumatic sternal plate is composed of a pressure plate 10 and a retaining device provided on the pressure plate 10. The pressure plate 10 can be applied to the blades of a retractor by way of the retaining device so that the pressure plate 10 is seated against the sagittal incision surface of one sternum half when a patient's sternum is spread by the retractor.

The retaining device is provided on the distal side of the pressure plate 10 and is therefore not visible in FIGS. 2A and 2B. The pressure plate 10 is composed of two parts 10A and 10B, which are hinged together by way of a joining element 10C. The retaining device is composed of two leaf springs which extend essentially parallel to the pressure plate 10 and are attached at the medial ends to the pressure plate 10. To be more precise, a leaf spring is provided on each part 10A and 10B of the two-part pressure plate 10. The joining element 10C comprises a base body 10C1 on which two round protrusions 10C2 and one elongated protrusion 10C3 are provided. Each of the two parts 10A and 10B of the pressure plate 10 has a hole, or a borehole, into which one of the round protrusions 10C2 of the joining element 10C is inserted. The medial end faces of the two parts 10A and 10B of the pressure plate 10 are shaped such that, in cooperation with the elongated protrusion 10C3, they allow a mutual relative rotation within a certain angular range. When the limit of this angular range is reached, the end faces of the two parts 10A and 10B are seated against the sides of the elongated protrusion 10C3 and prevent further rotation.

A third exemplary embodiment of the present invention is described hereafter with reference to FIGS. 3A and 3B. The third exemplary embodiment differs from the second exemplary embodiment in that no joining component is provided between the first and second parts 10A and 10B of the pressure plate 10. Instead, part 10A has an extension 11. One round cut-out 11A and two substantially elongated cut-outs 11B are provided in the extension 11 which is directed toward the other part 10B of the pressure plate 10. Part 10B of the pressure plate has a round protrusion 12A which, together with the cut-out 11A, forms a hinged joint for the two parts 10A and 10B of the pressure plate 10, and two further protrusions 12B, which are located in the elongated cut-outs 11B and in this way limit the maximum rotation of the two parts 10A and 10B with respect to each other.

A fourth exemplary embodiment of the present invention is described with reference to FIGS. 4A and 4B. The atraumatic sternal plate 100 according to the fourth exemplary embodiment has a multi-segmental design and is composed of one central element 100A and multiple further segments 100B. As is shown in FIG. 4B, one segment 100B has an extension 13 and a depression 14. The extension 13 is composed of an essentially trapezoidal web and a circular arc segment 13A. The depression 14 is composed of an essentially trapezoidal recess and a circular arc-shaped recess. An extension 13 is contained in a depression 14. The circular arc-shaped recess is wider than the circular arc segment 13A contained therein, so that rotation between two adjoining segments is possible within a certain angular range. To be more precise, the circular arc-shaped recess extends over a larger angular range than the circular arc segment 13A of extension 13. FIG. 4B shows the center plane of the atraumatic sternal plate. It is covered on both sides by covers. The covers on the proximal side form the pressure plate. The covers prevent the extensions 13 from moving out of the depressions 14. The retaining device is composed of two leaf springs (not shown in FIGS. 4A and 4B). One of these leaf springs, which corresponds to those of the third exemplary embodiment, is provided on each end segment. The central segment 100A differs from the other segments 100B in that the central segment 100A has no extension 13, but instead has two depressions 14.

In addition, each extension 14 is accommodated in the associated depression 13 in such a way that a certain friction fit exists between the segments, so that the angle between the two segments does not change as a result of the gravity of the sternal plate after the surgeon has adapted this angle to the shape of the patient's sternum or to the sagittal incision surface thereof. It is expedient for the surgeon to adapt this sternal plate to the shape of the sagittal incision surface of the patient, and then apply the blades of the retractor. However, it is also possible to apply the sternal plate first to the blades of the retractor, and to adapt the shape of the sternal plate while inserting the same into the opening of the sternum when the retractor is applied.

A fifth exemplary embodiment of the present invention is described with reference to FIGS. 5A and 5B. In this exemplary embodiment, the atraumatic sternal plate is likewise composed of a plurality of segments 200. Each segment 200 has a pressure plate 201, which, together with the pressure plates of the other segments, forms the overall pressure plate of the atraumatic sternal plate according to this exemplary embodiment. A joining element 205 is provided on the distal side of the segments 200. The joining element 205 is a flat strip that runs along the longitudinal direction of the sternal plate and is positioned perpendicularly to the pressure plate. The flat strip is dimensioned (thickness, width) such that that it is possible to adjust to the shape of the sagittal incision surface of the patient's sternum in the sagittal plane. Moreover, the flat strip is dimensioned such that it is able to transmit bending moments from one segment 200 to adjacent segments 200. For this purpose, the width of the strip is considerably greater than its thickness. The deformation of the flat strip can be an elastic deformation or a plastic deformation. In the latter case, the atraumatic sternal plate can be adapted to the patient prior to inserting the retractor into the opening of the sternum because, due to the plastic deformation, the adaptation is not lost when the atraumatic sternal plate is removed from the sternal opening and applied to the blades of the retractor.

Retaining devices of the sternal plate on a patient's sternum are shown with reference to FIGS. 7A through 7C. In FIG. 7A, the retaining device 20 is composed of a protrusion, which is provided on the pressure plate 10 and protrudes toward the proximal side of the plate. The protrusion 20 is intended to be seated against the outer surface of the patient's thorax, that is, against the soft tissue WG. The protrusion 20 can extend over the entire length of the pressure plate, or only over a certain region. The pressure plate can be designed to be single-piece, multi-piece or multi-segmental. In the latter case, a protrusion can be provided on each segment, or only on a single segment, or on selected segments. As is shown in FIG. 7B, the atraumatic pressure plate 10 can additionally be fixed on the patient's soft tissue WG with an adhesive strip K.

FIG. 7C shows a sternal plate in which the retaining device 20 is composed of a protrusion that is seated against the inside of the sternum S. To prevent the sternal plate from dropping into the patient's thorax in this case, the plate can be fixed on the soft tissue WG with an adhesive strip K. The adhesive strip is only one example of a known means of attachment. Other known means of attachment and types of attachment are also possible.

FIGS. 8A to 8C show additional retaining devices with the aid of which the sternal plate can be fixed on the sternum. In FIG. 8A, the pressure plate 50 has a protrusion 51 to be seated against the sternum S and a protrusion 52. The pressure plate, together with the protrusions 51 and 52, thus essentially forms a C shape. A leaf spring 53 is provided on the protrusion 52 in such a way that the spring can exert a pressure on the soft tissue WG with which to fix the sternal plate on the sternum S. The sternal plate can also have a multi-segmental design with such a retaining device. In this case, the cross-sections of the individual segments in particular may be different. In FIG. 8B, the leaf spring of FIG. 8A was replaced by a coil spring 54. The remaining configuration corresponds to that of FIG. 8A. FIG. 8C shows a retaining device with a spring 55. In contrast to FIG. 8A, in this case no protrusion is provided on the pressure plate on the side of the soft tissue. The pressure plate thus has an L-shaped cross-section, and the spring 55 is applied directly to the pressure plate.

A sixth exemplary embodiment of the present invention is described with reference to FIG. 9. The pressure plate is composed of two parts 60 and 62. A protrusion 61 or 63 is provided on each of these parts. The protrusion 61 is used to become seated against the inner surface of the sternum, while the protrusion 63 is used to become seated against the soft tissue WG. The part 62 is partially contained in the part 60, and the two parts 60 and 62 together form a telescopic pressure plate. With the aid of this telescopic pressure plate, the pressure plate can be adapted to the thickness of the patient's thorax, that is, to the width of the sagittal incision surface of the sternum. The part 62 can be contained in the part 60 so that the friction between these two parts maintains a set width of the pressure plate. However, it is also possible to position a tension spring between the two parts 60 and 62, which continuously pulls the part 62 toward the part 60. In this case, the sternum S and the soft tissue WG are clamped between the two protrusions 61 and 63. Such a design of the pressure plate is also possible and advantageous in the context of a multi-segmental pressure plate.

FIGS. 10A and 10B show simple options of how a means for hemostasis can be applied to a pressure plate. According to FIG. 10A, a body 80 which includes or holds the means for hemostasis is press fit into a recess provided in the pressure plate 70. According to FIG. 10, the pressure plate 90 does not have a depression, but instead has a protrusion 91 and a protrusion 92. The body 80, which includes or holds the means for hemostasis, is pushed behind a portion of the protrusion 91 and then clamped between the protrusion 91 and protrusion 92. Once again, there are numerous other options for the ways in which such a body 80 can be applied to a pressure plate.

Beyond the exemplary embodiments described herein, numerous further variations and modifications of the present invention will be apparent to a person skilled in the art from the present description, the claims and the figures.

In particular, it is possible to combine any manner of applying a means for hemostasis with any type of retaining device. A multi-segmental design of the pressure plate can also be combined with any retaining device, and even with the pressure plate that can be telescoped in the direction of its width. In the case of a multi-segmental pressure plate, a means for hemostasis can be provided either on each individual segment, or a body, preferably a non-woven fabric, a knitted fabric or a woven fabric, may be applied to the entire pressure plate by adhesion. Segmentation is also possible for all cross-sectional shapes of the pressure plate, that is, for a pressure plate having an L-shaped or a C-shaped cross-section, as well.

1. An atraumatic sternal plate for hemostasis for a retractor, having:
a pressure plate, and
a retaining device provided on the pressure plate, wherein
the pressure plate can be applied to the blades of a retractor by way of the retaining device so that the pressure plate is seated against the sagittal incision surface of one sternum half when a patient's sternum is spread by the retractor.

2. The atraumatic sternal plate according to claim 1, wherein
the pressure plate has an arched elongated shape so as to conform in the vertical direction to a curvature of the sagittal incision surface of the sternum half.

3. The atraumatic sternal plate according to claim 1, wherein
the retaining device is affixed to the distal side of the pressure plate.

4. The atraumatic sternal plate according to claim 3, wherein
the retaining device is composed of two leaf springs which extend essentially parallel to the pressure plate and are attached to the pressure plate at their medial ends.

5. The atraumatic sternal plate according to claim 4, wherein
the retaining device is composed of one leaf spring, the two free ends of which extend essentially parallel to the pressure plate and the central region of which is attached to the pressure plate.

6. The atraumatic sternal plate according to claim 4, wherein
the retaining device is composed of one leaf spring which extends essentially parallel to the pressure plate and is attached at its lateral ends to the pressure plate.

7. The atraumatic sternal plate for hemostasis, having:
a pressure plate and
a retaining device provided on the pressure plate, wherein
the pressure plate can be applied to a sternum half by the at least one retaining device so that the pressure plate is essentially seated against a sagittal incision surface of the sternum.

8. The atraumatic sternal plate according to claim 7, wherein
the pressure plate has an arched elongated shape so as to conform in the vertical direction to a curvature of the sagittal incision surface of the sternum half.

9. The atraumatic sternal plate for hemostasis according to claim 7, wherein
the retaining device has at least one protrusion on at least one longitudinal edge of the pressure plate, the protrusion projecting from the proximal side of the pressure plate.

10. The atraumatic sternal plate for hemostasis according to claim 9, wherein
the at least one protrusion extends essentially over the entire longitudinal edge of the pressure plate.

11. The atraumatic sternal plate for hemostasis according to claim 7, wherein
the retaining device has at least one protrusion on each of the two longitudinal edges, so that the sternal plate has a substantially C-shaped cross-section, at least one elastically compressible component being provided on the at least one protrusion, the component pressing the sternum from the inside and/or from the outside on the soft tissue thereabove when the sternal plate is used on the patient, thus holding the sternal plate in position.

12. The atraumatic sternal plate for hemostasis according to claim 7, wherein
the retaining device has at least one protrusion on each of the two longitudinal edges, so that the sternal plate has a substantially C-shaped cross-section, the pressure plate being telescopic in the transverse direction, so that the width or the height of the sternal plate can be adapted to the thickness of the patient's sternum and the soft tissue located thereabove.

13. The atraumatic sternal plate according to claim 1, wherein
the pressure plate is composed of at least two segments, the segments being rotatable or displaceable with respect to each other so as to be adaptable in the vertical direction to a curvature of the sagittal incision surface of the sternum half.

14. The atraumatic sternal plate according to claim 13, wherein
the rotation or displacement of the segments with respect to each other is subject to such friction that the arrangement of the segments cannot be altered exclusively by the effect of their own gravity.

15. The atraumatic sternal plate according to claim 1, wherein
the proximal side of the pressure plate is at least partially covered by a means for hemostasis, in particular by a collagen non-woven fabric.

16. The atraumatic sternal plate according to claim 15, wherein
the means for hemostasis is composed of a woven fabric, a non-woven fabric and/or another flat component, which is preferably covered or saturated with a chemical anticoagulant.

17. The atraumatic sternal plate according to claim 1, wherein
the pressure plate has at least one perforation and/or predetermined breaking point, by which at least one dimension of the pressure plate can be adapted to the size and shape of the patient's sternum or to the sagittal incision surface of the sternum.

18. The atraumatic sternal plate according to claim 17, wherein
at least one perforation and/or predetermined breaking point is provided between individual segments of the pressure plate.
